(19) United States
(12) Patent Application Publication
(10) Pub. No.: US 2026/0018299 A1
(43) Pub. Date: Jan. 15, 2026

(54) ARTIFICIAL INTELLIGENCE MODEL DEVICE OF ESTIMATING SURVIVAL RATES OF CRITICALLY ILL PATIENT

(71) Applicant: Kaohsiung Medical University, Kaohsiung (TW)

(72) Inventors: Ming-Ju Tsai, Kaohsiung (TW); Wei-An Chang, Kaohsiung (TW); Chih-Hung Cheng, Kaohsiung (TW); Yeong-Cheng Liou (Pao), Kaohsiung (TW); Chin-Jing Lee, Kaohsiung (TW); Chen-Yi Chou, Kaohsiung (TW); Chien-Chang Wu, Kaohsiung (TW); Deng-chiung Shih, Kaohsiung (TW); Chia-Lin Hsu, Kaohsiung (TW); Li-Li Wang, Chiayi County (TW)

(21) Appl. No.: 18/901,161

(22) Filed: Sep. 30, 2024

(30) Foreign Application Priority Data
Jul. 9, 2024 (TW) 113125637

(51) Int. Cl. G16H 50/30; G16H 50/20

(52) U.S. Cl. CPC G16H 50/30; G16H 50/20

(57) ABSTRACT

Provided is an artificial intelligence (AI) model device of estimating the survival rates of a critically ill patient, adapted to estimate short-, medium- and long-term survival rates of an intensive care unit (ICU) patient, including a monitoring module, a data processing module, an AI evaluation module, and a display module. Therefore, AI algorithmic computation is performed on the ICU patient (but not targeted at any specific disease) with an XGBoost-based AI algorithmic computation model according to the ICU patient's daily personal features, test report data, physiology data, and evaluation data to evaluate the ICU patient's 30-day, 60-day, and 90-day survival rates. The AI algorithmic computation model is effective in estimating the longer-term prognosis of a critically ill patient precisely and enabling an ICU team to allocate medical resources appropriately and communicate with the ICU patient's family members better.

### FIELD OF THE INVENTION

The present invention relates to an artificial intelligence model device of estimating the survival rates of a critically ill patient. More particularly, the present invention relates to a system of evaluating the prognosis of a critically ill patient admitted to an intensive care unit (ICU) and especially evaluating the critically ill patient's 30-day, 60-day and 90-day survival rates with an artificial intelligence (AI) algorithmic computation model developed by XGBoost.

### DESCRIPTION OF THE PRIOR ART

The most common conventional scoring system for use in estimating the survival rates of an ICU patient is Acute Physiology and Chronic Health Evaluation (APACHE) II or III. The most common conventional scoring system for use in estimating inpatient mortality rates is Sequential Organ Failure Assessment (SOFA). These scoring systems are developed decades ago, mainly with data of Caucasians. However, their applicability to the Asian population and their precision remains untested in the progressively improved critical care settings. On the other hand, not only is data collected by intensive care units (ICU) nowadays increasingly diverse and detailed, but machine-based analysis technology nowadays also cannot be more sophisticated. As a result, conventional critically ill patient prognosis scoring systems might not meet the ongoing demands for precisely-predicted, individual-oriented medical care nowadays.

A conventional technique of achieving early estimation of sepsis risks with a sepsis diagnosis model to predict an outbreak of sepsis within 12 hours before a clinical diagnosis, which enables medical professionals to treat patients earlier, greatly shortens the duration of hospitalization of critically ill ICU patients and reducing their mortality rates. However, the conventional technique estimates a specific disease risk but cannot estimate the survival rates of each ICU patient.

A conventional method, system, and computer-readable medium for use in evaluating the mortality rates of inpatients by: 1) integrating and analyzing a lot of input real-time continuous physiology signals (such as electrocardiogram); 2) performing advanced measurement (such as spectrum analysis) on the variability of the signals; 3) physiological organ function measurement (such as blood sugar); and 4) census and diagnosis-related mortality rate estimation factors, so as to achieve real-time, continuously-updated mortality risk estimation to assist physicians in providing medical care. However, the conventional method, system, and computer-readable medium use the hospital survival rate or ICU survival rate as a major therapy efficacy indicator but cannot estimate the longer-term prognosis of a critically ill patient.

The prognosis of a critically ill patient, for example, hospital mortality rate or ICU mortality rate, is most frequently evaluated or estimated with APACHE II, APACHE Ill or SOFA score. These scoring systems were established long time ago, and the data used in the statistical analyses to develop these scoring systems were collected more than 20 years ago. On the other hand, data recently collected for medical purposes surpasses its conventional counterparts in terms of diversity and details, not to mention that machine-based analysis technology nowadays cannot be more sophisticated. As a result, conventional prognosis scoring systems can no longer meet the ongoing demand for precisely-predicted, individual-oriented medical care. Therefore, it is necessary to provide a solution to the aforesaid drawbacks of the prior art and devise a way of precisely estimating the long-term prognosis of a critically ill patient, for example, the long-term survival rates of an ICU patient.

### BRIEF SUMMARY OF THE INVENTION

The main purpose of the present invention is to overcome the aforesaid drawbacks of the prior art and provide an artificial intelligence model device of estimating survival rates of a critically ill patient, targeted at each ICU patient (instead of a specific disease), and adapted to evaluate the 30-day, 60-day, and 90-day survival rates of an ICU patient with an XGBoost-based AI algorithmic computation model by performing AI algorithmic computation on the ICU patient's daily personal features, test report data, physiology data, and evaluation data. The AI algorithmic computation model is effective in estimating the longer-term prognosis of a critically ill patient precisely and enabling an ICU team to allocate medical resources appropriately and better communicate with the patient's family members.

To achieve the above purposes, the present invention is an artificial intelligence model device of estimating the survival rates of a critically ill patient, adapted to estimate the longer-term survival rates of an ICU (intensive care unit) patient, comprising:
a monitoring module for collecting clinical data about the ICU patient, the clinical data comprising personal features, test report data, physiology data, and evaluation data;
a data processing module for performing data quantification and normalization, including but not limited to missing value imputation, maximum calculation, minimum calculation, mean calculation, standard deviation calculation, median calculation, quartile deviation calculation, and data dimensionality reduction, on daily clinical data of the ICU patient to create related, applicable data;
an AI evaluation module for inputting the processed clinical data to a constructed AI algorithmic computation model to generate an evaluation result about the 30-day, 60-day, and 90-day survival rates of the ICU patient; and
a display module for receiving the evaluation result and displaying the estimated 30-day, 60-day, and 90-day survival rates of the ICU patient and a description of contributions of features in all categories.

In the embodiment of the present invention, the AI algorithmic computation model is an XGBoost-based machine learning model.

In the embodiment of the present invention, the AI algorithmic computation model entails inputting a lot of clinical data from the ICU patient, dividing the clinical data into a training dataset for machine learning and a testing dataset in a predetermined ratio, performing model training on the training dataset, performing testing on the testing dataset, undergoing verification to finalize the AI algorithmic computation model, and evaluating the performance of the AI algorithmic computation model with standard performance evaluation indicators.

In the embodiment of the present invention, the standard performance evaluation indicators include but are not limited to area under the receiver operating characteristic curve (AU-ROC), F1 score, precision, recall and accuracy.

In the embodiment of the present invention, the personal features and the evaluation data at least include categories as followings:
first category: pregnancy state, age, sex, body height, body weight, body mass index (BMI), and smoking history;
second category: Where was the patient before being admitted to the ICU? Did the patient receive cardiopulmonary resuscitation before being admitted to the ICU? Did a cardiac arrest event occur before the patient was admitted to the ICU? and
third category: Did the patient receive elective surgery before being admitted to the ICU? Was admission to the ICU planned? Did the patient receive intubation for mechanical ventilation? How was the partial pressure of inspired oxygen (FiO₂)?

In the embodiment of the present invention, the physiology data and the evaluation data are physiology monitoring data collected within 24 hours, and the test report data is the latest blood test data collected within 48 hours.

In the embodiment of the present invention, the physiology data and the evaluation data at least include categories as follows:
first category: body temperature, heart rate, respiratory rate (and its oxygen utilization or mechanical ventilation settings), systolic blood pressure, diastolic blood pressure, mean arterial pressure, and Glasgow Coma Scale (GCS); and
second category: volume of urine excreted in a 24-hour period.

In the embodiment of the present invention, the test report data comprises white blood cell count, hemoglobin, platelet count, blood sodium level, blood potassium level, blood creatinine level (mg/dL), estimated glomerular filtration rate (eGFR), blood urea nitrogen (BUN) (mg/dL), serum albumin level (g/dL), blood bilirubin level (mg/dL), blood sugar level (mg/dL), blood lactic acid level, partial pressure of carbon dioxide in arterial blood (PaCO₂), partial pressure of oxygen in arterial blood (PaO₂), and arterial pH.

### BRIEF DESCRIPTION OF THE DRAWINGS

The present invention will be better understood from the following detailed description of preferred embodiments of the invention, taken in conjunction with the accompanying drawings, in which

FIG. 1 is a schematic view of how the survival rates of a critically ill patient are estimated by an artificial intelligence (AI) model device according to the present invention;

FIG. 2 is a histogram of various estimated survival rates of an ICU patient, including ICU survival rate, hospital survival rate (H), and 30-, 60-, 90-day survival rates. The 30-, 60-, 90-day survival rates are estimated by an AI algorithmic computation model of the present invention;

FIG. 3A is a receiver operating characteristic (ROC) curve showing the performance of various estimating methods for 30-day survival rate, suggesting the AI algorithmic computation model of the present invention, in either testing (test), training (train), or validation (val) dataset, surpasses conventional scoring systems (AHACHE II and SOFA);

FIG. 3B is an ROC curve showing the performance of various estimating methods for 60-day survival rate, suggesting the AI algorithmic computation model of the present invention, in either testing (test), training (train), or validation (val) dataset, surpasses conventional scoring systems (AHACHE II and SOFA);

FIG. 3C is an ROC curve showing the performance of various estimating methods for 90-day survival rate, suggesting the AI algorithmic computation model of the present invention, in either testing (test), training (train), or validation (val) dataset, surpasses conventional scoring systems (AHACHE II and SOFA);

FIG. 4A is a graphical representation of the feature values used by the AI algorithmic computation model in estimating 30-day survival rate against the SHAP (SHapley Additive explanations) values;

FIG. 4B is a graphical representation of the feature values used by the AI algorithmic computation model in estimating 60-day survival rate against the SHAP values; and

FIG. 4C is a graphical representation of the feature values used by the AI algorithmic computation model in estimating 90-day survival rate against the SHAP values.

### DESCRIPTION OF THE PREFERRED EMBODIMENTS

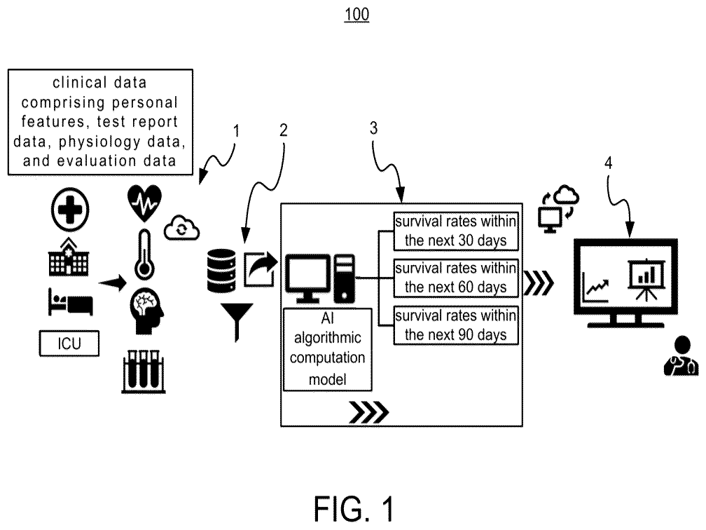

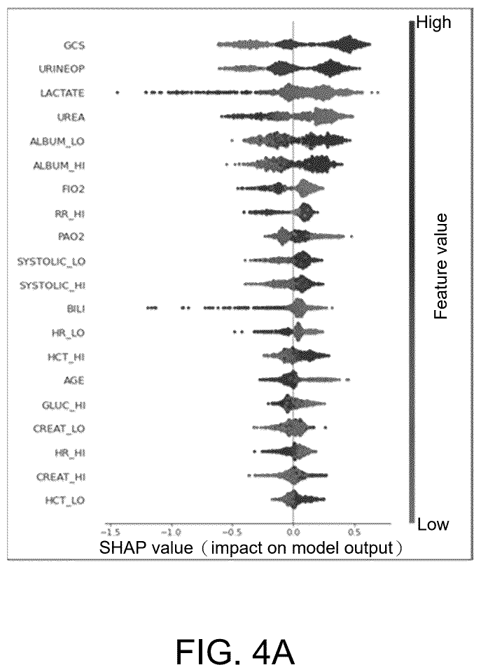

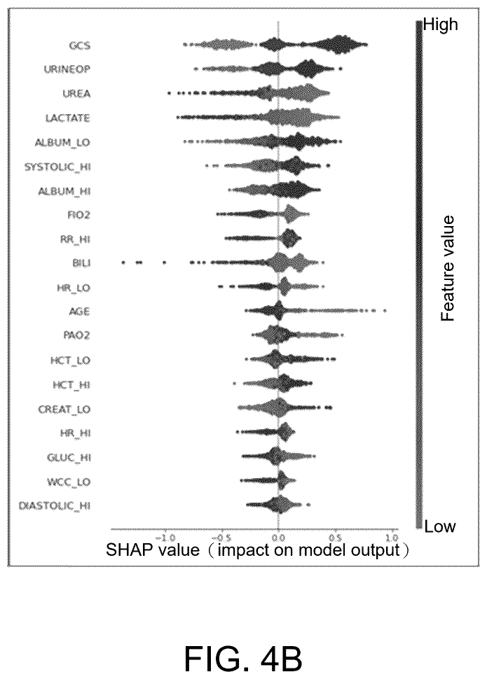

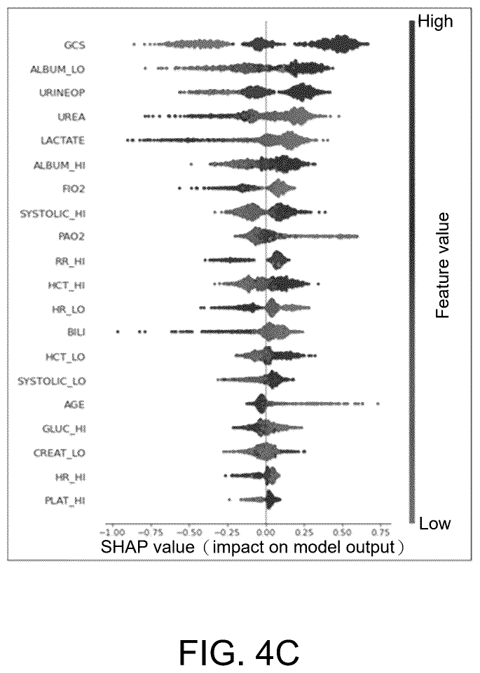

Referring to FIG. 1˜FIG. 4C, there are shown a schematic view of how the survival rates of a critically ill patient are estimated by an AI model device according to the present invention, a histogram of various estimated survival rates of an ICU patient, including ICU survival rate, hospital survival rate, and 30-, 60-, 90-day survival rates estimated by an AI algorithmic computation model of the present invention, ROC curves showing the performances of various estimating methods for 30-, 60-, and 90-day survival rates, and graphical representations of the feature values used by the AI algorithmic computation model in estimating 30-, 60-, and 90-day survival rates against the SHAP values. As shown in the diagrams, the present invention is an AI model device 100 of estimating the survival rates of a critically ill patient, adapted to estimate the longer-term survival rates of an ICU (intensive care unit) patient, comprising a monitoring module 1, a data processing module 2, an artificial intelligence (AI) evaluation module 3, and a display module 4.

The monitoring module 1 collects clinical data about the patient admitted to ICU.

Then, the data processing module 2 performs data quantification and normalization, including but not limited to missing value imputation, maximum calculation, minimum calculation, mean calculation, standard deviation calculation, median calculation, quartile deviation calculation, and data dimensionality reduction, on daily clinical data of the ICU patient.

Next, the AI evaluation module 3 generates evaluation results of estimating the 30-day, 60-day, and 90-day survival rates of the ICU patient with an AI algorithmic computation model, which is an XGBoost-based machine learning model.

After that, the display module 4 displays the evaluation results of the estimated 30-day, 60-day, and 90-day survival rates of the ICU patient and a description of contributions of features in all categories to assist in explaining prognosis to the ICU patient and the ICU patient's relatives, planning treatments, and allocating medical resources.

Therefore, the monitoring module 1, data processing module 2, artificial intelligence (AI) evaluation module 3, and display module 4 together constitute a novel AI model device 100 of estimating the survival rates of a critically ill patient.

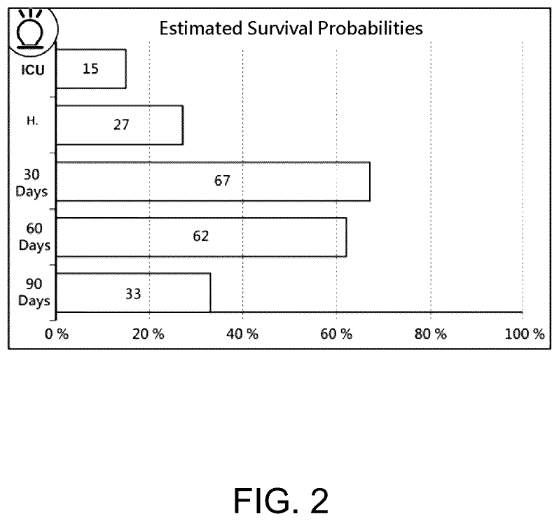

The AI model device 100 of estimating the survival rates of a critically ill patient is targeted at each ICU patient instead of any specific disease. The operation of the AI model device 100 is described below. First, the monitoring module 1 collects the ICU patient's clinical data, including personal features, test report data, physiology data, and evaluation data. Then, the data processing module 2 performs data quantification and normalization on the clinical data to create related, applicable data. Next, the AI evaluation module 3 inputs the processed clinical data to a constructed AI algorithmic computation model and generate evaluation results of estimating the 30-day, 60-day, and 90-day survival rates of the ICU patient. Finally, the display module 4 receives the evaluation results and displays the estimated 30-day, 60-day, and 90-day survival rates of the ICU patient, as shown in FIG. 2, but the present invention is not limited thereto. Therefore, the AI algorithmic computation model is effective in estimating the longer-term prognosis of a critically ill patient precisely and enabling the ICU team to allocate medical resources appropriately and communicate with the ICU patient's family members better.

Therefore, the present invention is applicable to medical treatment, and the AI model device of the present invention is applicable to each ICU patient, using the AI algorithmic computation model to perform AI algorithmic computation on the ICU patient's daily physiology data, test report data, and evaluation data, evaluate the ICU patient's 30-day, 60-day, and 90-day survival rates, quantify severity of diseases, achieve precise estimation of survival rates, and enable the ICU team to provide medical interventions as early as possible so as to assist with explaining prognosis to the ICU patient and the ICU patient's relatives, planning treatments, and allocating medical resources.

In a preferred specific embodiment of the present invention, the AI algorithmic computation model entails inputting a lot of clinic data from an ICU patient, dividing the clinic data into a training dataset for machine learning and a testing dataset in a predetermined ratio, performing model training on the training dataset, performing testing on the testing dataset, undergoing verification to finalize the AI algorithmic computation model, and evaluating the performance of the AI algorithmic computation model with standard performance evaluation indicators.

In a preferred specific embodiment of the present invention, the standard performance evaluation indicators include but are not limited to area under the receiver operating characteristic curve (AU-ROC), F1 score, precision, recall, and accuracy.

In a preferred specific embodiment of the present invention, an XGBoost-based AI algorithmic computation model is used to analyze clinical data, such as personal features, test report data, physiology data, and evaluation data, to obtain 16189 samples, divide the samples into 12951 pieces of training dataset and 3238 pieces of testing dataset for undergoing training and testing respectively and verification. The present invention involves using variables, such as personal features, physiology data, evaluation data, and test report data.

In a preferred specific embodiment of the present invention, the personal features and the evaluation data include but are not limited to categories as follows:
(a) pregnancy state, age, sex, body height, body weight, body mass index (BMI), and smoking history;
(b) Where was the patient before being admitted to the ICU? Did the patient receive cardiopulmonary resuscitation before being admitted to the ICU? Did a cardiac arrest event occur before being admitted to the ICU? and
(c) Did the patient receive elective surgery before being admitted to the ICU? Was admission to the ICU planned? Did the patient receive intubation for mechanical ventilation? How was the partial pressure of inspired oxygen (FiO₂)?

In a preferred specific embodiment of the present invention, the physiology data and the evaluation data are physiology monitoring data collected within 24 hours, including categories as follows:
(a) body temperature, heart rate, respiratory rate (and its oxygen utilization or mechanical ventilation settings), systolic blood pressure, diastolic blood pressure, mean arterial pressure, and Glasgow Coma Scale (GCS); and
(b) volume of urine excreted in a 24-hour period.

In a preferred specific embodiment of the present invention, the test report data is the latest piece of blood test data collected within 48 hours, including:
(a) white blood cell count, hemoglobin, platelet count, blood sodium level, blood potassium level, blood creatinine level (mg/dL), estimated glomerular filtration rate (eGFR), blood urea nitrogen (BUN) (mg/dL), serum albumin level (g/dL), blood bilirubin level (mg/dL), blood sugar level (mg/dL), blood lactic acid level, partial pressure of carbon dioxide in arterial blood (PaCO₂), partial pressure of oxygen in arterial blood (PaO₂), and arterial pH.

An embodiment is described below for exemplary purposes to enable persons skilled in the art to gain insight into the details and principles of the present invention. The embodiment is not restrictive of the claims of the present invention.

### Purposes

Intensive care units (ICU) nowadays are a good source of delicate, intricate clinical data conducive to the development of precise evaluation models through integrated learning technology. The present invention involves constructing an AI algorithmic computation model for use in estimating an ICU patient's 30-day, 60-day and 90-day survival rates.

### Materials and Method

The present invention involves collecting an ICU patient's clinical data, including personal features, test report data, physiology data, and evaluation data, retrospectively and developing an-XGBoost based AI algorithmic computation model for estimating 30-day, 60-day and 90-day survival rates. The performance of the AI algorithmic computation model is evaluated in terms of AU-ROC, precision, F1 score, accuracy and recall. The AU-ROC of the AI algorithmic computation model is compared with the AU-ROC of Acute Physiology and Chronic Health Evaluation (APACHE) II and Sequential Organ Failure Assessment (SOFA) score.

### Result

The present invention involves collecting 16189 pieces of data pertaining to critically ill patients, including 12951 pieces in the training dataset and 3238 pieces in the testing dataset, analyzing clinical data, such as personal features, test report data, physiology data, and evaluation data, and evaluating 30-day, 60-day and 90-day survival rates with an XGBoost-based AI algorithmic computation model. As shown in Table 1, regarding the performance of the AI algorithmic computation model and conventional scoring systems in estimating 30-day, 60-day and 90-day survival rates in the testing dataset, the AI algorithmic computation model for estimating the 30-day, 60-day and 90-day survival rates has ratings of 0.83, 0.82 and 0.81 in terms of AU-ROC respectively, ratings of 0.90, 0.88 and 0.88 in terms of F1 score respectively, ratings of 0.83, 0.80 and 0.80 in terms of accuracy respectively, ratings of 0.96, 0.93 and 0.93 in terms of recall respectively, and ratings of 0.85, 0.84 and 0.83 in terms of precision respectively. The AI algorithmic computation model has higher ratings than APACHE II and SOFA score in terms of AU-ROC (30-day survival rates: 0.83 vs. 0.80 vs. 0.76; 60-day survival rates: 0.82 vs. 0.78 vs. 0.75; 90-day survival rates: 0.81 vs. 0.78 vs. 0.74).

TABLE 1

| | AI algorithmic computation model of the present | | | | |
|---|---|---|---|---|---|
| | invention | | | | |
| model | | | | AU-ROC | AU-ROC |
| performance | | | | (training | (testing |
| indicator | precision | recall | accuracy | dataset) | dataset) |
| 30-day survival | 0.85 | 0.96 | 0.83 | 0.94 | 0.83 |
| rate | | | | | |
| 60-day survival | 0.84 | 0.93 | 0.80 | 0.95 | 0.82 |
| rate | | | | | |
| 90-day survival | 0.83 | 0.93 | 0.80 | 0.92 | 0.81 |
| rate | | | | | |

| rating system | | |
|---|---|---|
| performance | APACHE II | SOFA |
| indicator | AU-ROC | AU-ROC |
| 30-day survival | 0.80 | 0.76 |
| rate | | |
| 60-day survival | 0.78 | 0.75 |
| rate | | |
| 90-day survival | 0.78 | 0.74 |
| rate | | |

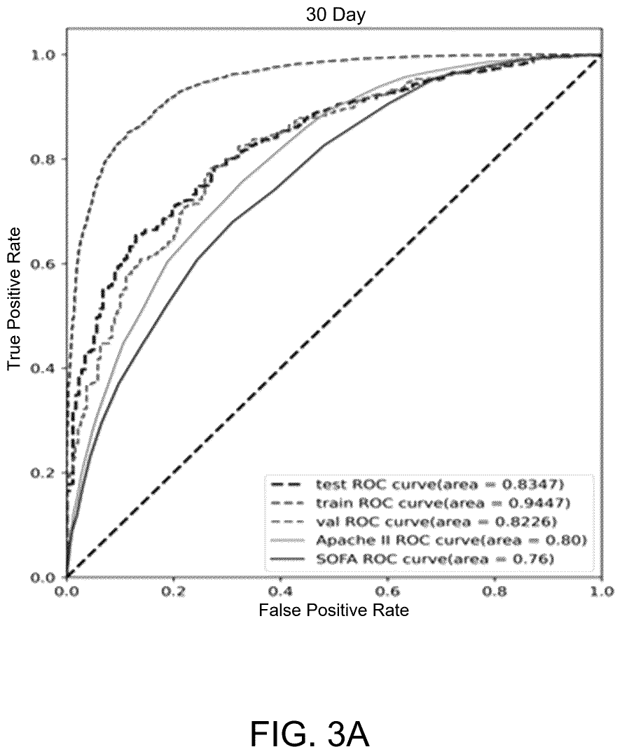

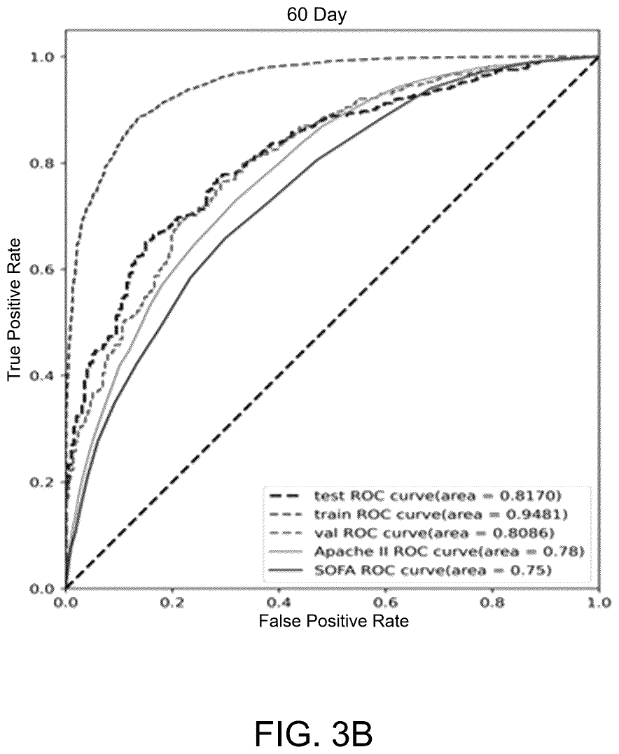

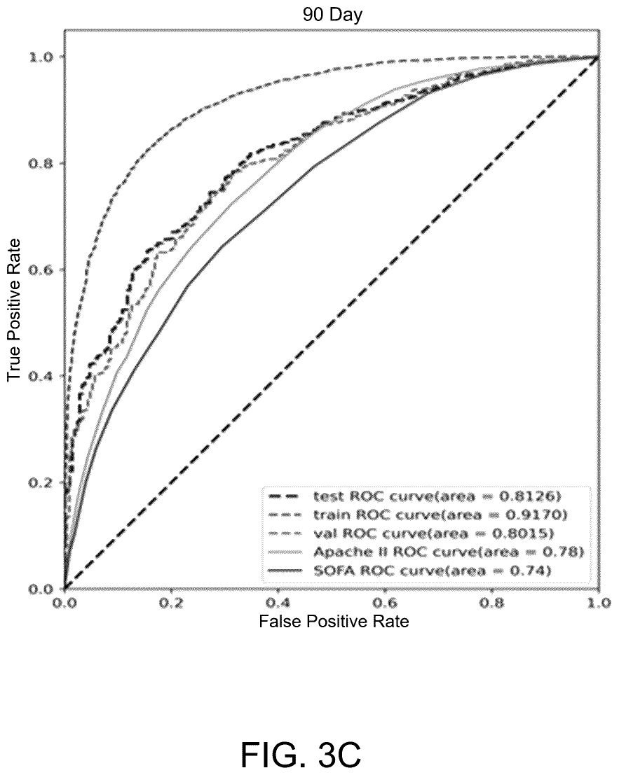

FIG. 3A˜FIG. 3C depict the performance of an AI algorithmic computation model of the present invention and conventional scoring systems (APACHE II and SOFA score) in estimating 30-day (FIG. 3A), 60-day (FIG. 3B), and 90-day (FIG. 3C) survival rates in terms of ROC curves. FIG. 4A˜FIG. 4C show feature values used by the AI algorithmic computation model of the present invention in estimating 30-day (FIG. 4A), 60-day (FIG. 4B), and 90-day (FIG. 4C) survival rates in terms of SHAP (SHapley Additive explanations) values respectively.

### Conclusion

The AI algorithmic computation model outperforms APACHE II and SOFA in estimating the 30-day, 60-day and 90-day survival rates of an ICU patient. The AI algorithmic computation model is effective in estimating the longer-term prognosis of a critically ill patient precisely and enabling an ICU team to allocate medical resources appropriately and communicate with the ICU patient's family members better.

In conclusion, the present invention is an AI model device of estimating the survival rates of a critically ill patient, adapted to effectively overcome drawbacks of the prior art, targeted at each ICU patient instead of any specific disease, and adapted to perform AI algorithmic computation on the ICU patient's daily personal features, test report data, physiology data, and evaluation data with an XGBoost-based AI algorithmic computation model to evaluate the 30-day, 60-day, and 90-day survival rates of the ICU patient. The AI algorithmic computation model is effective in estimating the longer-term prognosis of a critically ill patient precisely and enabling the ICU team to allocate medical resources appropriately and communicate with the ICU patient's family members better.

The preferred embodiments herein disclosed are not intended to unnecessarily limit the scope of the invention. Therefore, simple modifications or variations belonging to the equivalent of the scope of the claims and the instructions disclosed herein for a patent are all within the scope of the present invention.

What is claimed is:

1. An artificial intelligence (AI) model device of estimating survival rates of a critically ill patient, adapted to estimate longer-term survival rates of an ICU (intensive care unit) patient, comprising:
a monitoring module for collecting clinical data about the ICU patient, the clinical data comprising personal features, test report data, physiology data, and evaluation data;
a data processing module for performing data quantification and normalization, including but not limited to missing value imputation, maximum calculation, minimum calculation, mean calculation, standard deviation calculation, median calculation, quartile deviation calculation, and data dimensionality reduction, on daily clinical data of the ICU patient to create related, applicable data;
an AI evaluation module for inputting the processed clinical data to a constructed AI algorithmic computation model to generate evaluation results about the 30-day, 60-day, and 90-day survival rates of the ICU patient; and
a display module for receiving the evaluation result and displaying the estimated 30-day, 60-day, and 90-day survival rates of the ICU patient and a description of contributions of features in all categories.

2. The AI model device of estimating survival rates of a critically ill patient according to claim 1, wherein the AI algorithmic computation model is an XGBoost-based machine learning model.

3. The AI model device of estimating survival rates of a critically ill patient according to claim 1, wherein the AI algorithmic computation model entails inputting a lot of clinical data from the ICU patient, dividing the clinical data into a training dataset for machine learning and a testing dataset in a predetermined ratio, performing model training on the training dataset, performing testing on the testing dataset, undergoing verification to finalize the AI algorithmic computation model, and evaluating the performance of the AI algorithmic computation model with standard performance evaluation indicators.

4. The AI model device of estimating survival rates of a critically ill patient according to claim 3, wherein the standard performance evaluation indicators include but are not limited to area under the receiver operating characteristic curve (AU-ROC), F1 score, precision, recall, and accuracy.

5. The AI model device of estimating survival rates of a critically ill patient according to claim 1, wherein the personal features and the evaluation data at least include categories as follows:
first category: pregnancy state, age, sex, body height, body weight, body mass index (BMI), and smoking history;
second category: Where was the patient before being admitted to the ICU? Did the patient receive cardiopulmonary resuscitation before being admitted to the ICU? Did a cardiac arrest event occur before being admitted to the ICU? and
third category: Did the patient receive elective surgery before being admitted to the ICU? Was admission to the ICU planned? Did the patient receive intubation for mechanical ventilation? How was the partial pressure of inspired oxygen (FiO₂).

6. The AI model device of estimating survival rates of a critically ill patient according to claim 1, wherein the physiology data and the evaluation data is physiology monitoring data collected within 24 hours, and the test report data is the latest piece of blood test data collected within 48 hours.

7. The AI model device of estimating survival rates of a critically ill patient according to claim 1, wherein the physiology data and the evaluation data at least include categories as follows:
first category: body temperature, heart rate, respiratory rate (and its oxygen utilization or mechanical ventilation state), systolic blood pressure, systolic blood pressure, mean arterial pressure, and Glasgow Coma Scale (GCS); and
second category: volume of urine excreted in a 24-hour period.

8. The AI model device of estimating survival rates of a critically ill patient according to claim 1, wherein the test report data includes white blood cell count, hemoglobin, platelet count, blood sodium level, blood potassium level, blood creatinine level (mg/dL), estimated glomerular filtration rate (eGFR), blood urea nitrogen (BUN) (mg/dL), serum albumin level (g/dL), blood bilirubin level (mg/dL), blood sugar level (mg/dL), blood lactic acid level, partial pressure of carbon dioxide in arterial blood (PaCO₂), partial pressure of oxygen in arterial blood (PaO₂), and arterial pH.
